(19) United States
(12) Patent Application Publication
(10) Pub. No.: US 2009/0208429 A1
(43) Pub. Date: Aug. 20, 2009

(54) GLOW AND SUNLESS TANNING VIA A HEATED FLOWABLE COSMETIC TRANSFERRED ONTO SKIN

(75) Inventors: Stephen Roy Barrow, Trumbull, CT (US); Philip Edward Miner, Newtown, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., d/b/a UNILEVER, Englewood Cliffs, NJ (US)

(21) Appl. No.: 12/032,736

(22) Filed: Feb. 18, 2008

(51) Int. Cl. A61K 8/35; A61Q 17/04

(52) U.S. Cl. 424/59

(57) ABSTRACT

A product and method for tanning or imparting glow to skin is herein described. The product is a package with associated instructions for applying a tanning cosmetic composition to skin dispensed from a heating device. The heated cosmetic composition imparts a more rapid development of color than an identical composition applied at room temperature.

### BACKGROUND OF THE INVENTION

1. Field of the Invention

The invention concerns a product for imparting glow and/or sunless tanning to skin that includes a flowable cosmetic composition formulated with a skin tanning agent and dispensed from a heating device.

2. The Related Art

Today there is a great health concern with natural tanning through sunlight. Ultraviolet radiation from the sun is considered to be a leading factor in causing skin cancer. Even if not lethal, ultraviolet radiation has been acknowledged as accelerating aging and wrinkling processes on the skin.

Beyond health concerns, there are obvious practical reasons against natural tanning. Foremost is the reason that in many areas of the globe and during all but summertime, there is insufficient sunlight available to accomplish a natural tan.

While some choose artificial sunlight as an alternative to exposure to natural sunlight, and as a potentially less risky means of obtaining a tan, this option too is not always suitable; hence, other options for skin tanning are desirable. One of these options, to which people are turning in increasing numbers, are the various “sunless tanning compositions” that can be used to impart a tan to human skin without the necessity of exposing the skin to natural, or artificial, sunlight. Such compositions contain, as their active agent, one or more of the available self tanning agents, including dihydroxyacetone (“DHA,” 1,3-dihydroxy-2-propanone).

DHA, currently the most widely used of the self tanning agents, is believed to exert its effect through interactions between its hydroxyl groups and the amino groups of amino acids and peptides naturally occurring in the hydrolipid pellicle and first layers of the stratum corneum of the skin. Such Maillard reactions are believed (see, e.g., Bobin et al., J. Soc. Cosmet. Chem. 35: 255 (1984)) to lead to formation of brown pigments in the skin, thereby giving it an appearance similar to that of a naturally obtained tan.

Although there has been great progress in sunless self-tanning as noted above, considerable further progress is needed to increase speed of coloration and achieve a coloration even closer to a natural tan. Furthermore, many of the known self-tanning compositions have storage stability problems. DHA can rapidly decompose in many formulations. Streaking is also a problem faced by the art. By the term “streaking” is meant noneven deposition on the akin; the tan coloration tends to migrate along an outer perimeter as a result of the formula being rubbed around upon the skin.

New approaches are required to address one or more of the problems with self-tanning compositions. Thus, the present invention is directed to both a product and method which solves the problem of increasing rates of coloration. In certain embodiments the invention may also impart a more natural hue to the skin and/or overcome streaking.

### SUMMARY OF THE INVENTION

A skin tanning product delivered through a heating device is provided which includes:
(i) a cosmetic composition having from about 0.1 to about 20% by weight of a tanning agent in a cosmetically acceptable carrier;
(ii) a package containing the cosmetic composition; and
(iii) instructions associated with the package describing use of the cosmetic composition which includes charging the cosmetic composition into the heating device, applying heat to the composition, thereafter activating a dispensing mechanism associated with the device and transferring dispensed heated composition to a human body.

Further, a method for tanning or imparting glow to skin is provided which includes:
(i) a cosmetic composition having from about 0.1 to about 20% by weight of a tanning agent in a cosmetically acceptable carrier;
(ii) providing a heating device including a chamber for receiving the cosmetic composition, a heating element for imparting heat to the cosmetic composition, and an outlet for dispensing heated cosmetic composition; and
(iii) dispensing from the heating device and applying the heated cosmetic composition onto the skin.

### DETAILED DESCRIPTION OF THE INVENTION

Now it has been found that tanning agent containing cosmetic compositions when applied at elevated temperature to the skin result in a faster development of coloration. The resultant coloration provides a glow to skin. Higher concentrations of tanning agent go beyond glow to impart sunless browning with the richness of natural sunlight exposure.

Tanning agents of the present invention are materials which when applied to human skin will react with amino acids of the skin so as to form pigmented species. These reactions give skin a brown appearance similar to a color obtained by exposure to sunlight. These materials may be alpha-hydroxyaldehydes and ketones, glyceraldehyde, troxerutin and related alcohol aldehydes, various indoles, imidazoles and derivatives thereof. Illustrative but not limiting are dihydroxyacetone, melanin, mahakanni (eclipta alba), methyl glyoxal, erythrulose, alloxan, 2,3-dihydroxysuccindialdehyde and mixtures thereof. More preferred is dihydroxyacetone.

Amounts of the tanning agent may range from about 0.1 to about 20%, preferably from about 0.5 to about 10%, more preferably from about 0.8 to about 5%, and optimally from 1 to 2% by weight of the cosmetic composition. Of particular usefulness is a combination of dihydroxyacetone and erythrulose in a relative weight ratio of 6:1 to 1:2, preferably a ratio from 4:1 to 2:1.

Formulations intended for imparting glow (synonymous with radiance), utilize tanning agent at lower levels such as from 0.5 to 2.5% by weight. Sunless tanning to a relatively dark shade ordinarily requires the tanning agent to be at the higher levels from 3 to 20% by weight.

Dispensing devices for heating cosmetic fluid compositions have been described in U.S. Pat. No. 6,216,911 B1; U.S. Patent Application Publication 2002/0108965 A1 and U.S. Pat. No. 6,056,160, the specifications of which are herein incorporated by reference. Also there are commercial devices available. One device is sold by Conair Corporation of Stamford, Conn. under the designation HLD 31 and HLD 20, and another by New Sensations LLC of Englewood, Colo. under the brand New Sensation Lotion Spa.

Heating devices of the present invention are best operated to deliver a composition that exhibits a dispensed temperature between about 30° to about 60° C., more preferably from 38° C. to 54° C., even more preferably from 40° to 49° C. and optimally from 42° to 46° C.

Compositions of the present invention will also include a cosmetically acceptable carrier. Water is the most preferred carrier. Amounts of water may range from about 1 to about 99%, preferably from about 5 to about 90%, more preferably from about 35 to about 70%, optimally between about 40 and about 60% by weight. Ordinarily the compositions will be water and oil emulsions of the W/O or O/W variety.

Other cosmetically acceptable carriers may include mineral oils, silicone oils, synthetic or natural esters, fatty acids and alcohols and humectants. Amounts of these materials may range from about 0.1 to about 50%, preferably from about 0.1 to about 30%, more preferably from about 1 to about 20% by weight of the composition.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, preferably from about 4 to about 5, silicon atoms.

Linear volatile silicone materials generally have viscosities less than about 5 centistokes at 25° C. while cyclic materials typically have viscosities of less than about 10 centistokes.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C.

Among suitable esters are:
(1) Alkenyl or alkyl esters of fatty acids having 10 to 20 carbon atoms. Examples thereof include isopropyl palmitate, isopropyl isostearate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxy-ethylene sorbitan fatty acid esters are satisfactory polyhydric alcohol esters.
(4) Wax esters such as beeswax, spermaceti, myristyl myristate, stearyl stearate.
(5) Sterols esters, of which soya sterol and cholesterol fatty acid esters are examples thereof.

Fatty acids having from 10 to 30 carbon atoms may be included in the compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be included in the compositions of this invention. The humectant aids in increasing the effectiveness of the emollient, reduces scaling, stimulates removal of built-up scale and improves skin feel. Typical polyhydric alcohols include glycerol (also known as glycerin), polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably glycerin. The amount of humectant may range anywhere from 0.5 to 30%, preferably between 1 and 15% by weight of the composition.

Emulsifiers may be present in cosmetic compositions of the present invention. Total concentration of the emulsifier may range from about 0.1 to about 40%, preferably from about 1 to about 20%, optimally from about 1 to about 5% by weight of the total composition. The emulsifier may be selected from the group consisting of anionic, nonionic, cationic and amphoteric actives. Particularly preferred nonionic surfactants are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition.

Thickening agents may be included in compositions of the present invention. Particularly useful are the polysaccharides. Examples include starches, natural/synthetic gums and cellulosics. Representative of the starches are chemically modified starches such as aluminum starch octenylsuccinate. Suitable gums include xanthan, scierotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are still a further class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature of Sodium Acrylate/Sodium Acryloyidimethyl Taurate and Acryloyl Dimethyltaurate/Vinyl Pyrrolidone Copolymer.

Amounts of the thickener may range from about 0.001 to about 5%, preferably from about 0.1 to about 2%, optimally from about 0.2 to about 0.5% by weight.

Colorants, fragrances and abrasives may also be included in compositions of the present invention. Each of these substances may range from about 0.05 to about 5%, preferably between 0.1 and 3% by weight.

As used herein, “color” is a general term intended to cover human perception of color and includes variations in lightness/darkness and/or variations in hue.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

The following Examples will more fully illustrate the embodiments of this invention. All parts, percentages and proportions referred to herein and in the appended claims are by weight unless otherwise indicated.

### EXAMPLE 1

A typical body lotion employing a tanning agent (dihydroxyacetone) according to this invention is disclosed in Table 1.

TABLE 1

| Component | Weight % |
|---|---|
| Stearic Acid | 2.34 |
| Glyceryl Monostearate/Stearamide AMP | 1.38 |
| Glycerol Monostearate | 0.65 |
| Cetyl Alcohol | 0.37 |
| Petrolatum | 1.25 |
| Isopropylmyristate | 1.30 |
| Disodium EDTA | 0.05 |
| Thickening Agent | 1.75 |
| Glycerin | 10.00 |
| Titanium Dioxide | 0.10 |
| Triethanolamine (99%) | 0.70 |
| Glydant Plus ® | 0.09 |
| DMDM Hydantoin | 0.17 |
| Silicone 50 ct | 1.50 |
| Silicone DC 1501 ® | 0.50 |
| Dihydroxyacetone | 1.50 |
| Fragrance | 0.30 |
| Water | Balance |

The lotion of Table 1 is formulated in the following manner. A reactor is charged with the deionized water and disodium EDTA. Heat is applied till 60° C. in combination with stirred mixing. Thickening agent is added to the reactor and the temperature maintained at 77-80° C. for 10 to 15 minutes. In a separate vessel, the oil phase components are added. Light mixing of the batch is performed with heating in a water bath to 75-77° C. The water reactor is maintained at 60-65° C. and slow addition occurred for glycerin, titanium dioxide and triethanolamine. Continuous mixing is done until the aqueous system is uniform. Very slowly the oil phase is added to the water phase at 75-77° C. under moderate mixing. After full emulsification, the batch is agitated for a further 5 minutes. Thereupon the resultant emulsion is homogenized using an ARDE Barenco® apparatus for 20-30 seconds at 35%. The resultant system is then topped with further deionized water. Cooling is then begun with a large sweep (50 rpm) mixer. Preservatives Glydant Plus® and DMDM Hydantoin is then added with the batch held at 50-55° C. Thereafter a slurry of dihydroxyacetone in the silicone oils is added to the batch. At a temperature of 45-50° C., the fragrance is charged to the reactor. Heating is then discontinued and mixing stopped when the temperature reaches 38-40° C.

The lotion is charged into a Conair HLD 31® Lotion Dispenser. The lotion is heated to a temperature between 38° C. and 54° C. A measured amount of sample is then dispensed from the heated dispenser. This sample amount is placed upon a panelist's forearm. On an opposite forearm of the panelist, a cool (25° C.) identical sample amount of lotion is placed for evaluation. Within 5 minutes of deposition on the forearm, the pre-heated sample will deliver a bronze glow. The non-heated sample left on the panelist's other forearm will require more than double the time to achieve similar coloration.

### EXAMPLE 2

An in vitro comparative experiment was conducted to evaluate the rate of glow or tanning between a pre-heated lotion and an identical but non-heated sample. The tests were conducted on the lotion formula outlined in Example 1. The procedure involved utilizing a No. 22 wire rod to cast a 56 micron wet film of the lotion on a 6×6 inch square (2.54 cm per inch) glass plate. A Minolta®® CR-21 Chroma Meter was used to measure the film's absolute chromaticity at 25° and 45° C. in 5 minute intervals. Chromaticity employed the L*a*b* color system.

This color system is known as the Commission Internationale de l'Eclairage (CIE) L*a*b* color system, where:

L*=black to white (luminance) from 0 to 100 [L*=0 represents black]

a*=green to red from −60 to +60

b*—blue to yellow from −60 to +60

Results of the test are reported in Table 2.

TABLE 2

| | |
|---|---|
| a* Scale | b* Scale |

| Time (min.) | 25° C. | 45° C. | 25° C. | 45° C. |
|---|---|---|---|---|
| 0 | −0.90 | −0.96 | −4.50 | −0.30 |
| 5 | −0.65 | −0.58 | −3.00 | −1.90 |
| 10 | −0.59 | −0.55 | −1.90 | −1.45 |
| 15 | −0.57 | −0.54 | −1.50 | −1.40 |
| 20 | −0.54 | −0.54 | −1.40 | −1.40 |

The a* scale shows the development of “−a” (green) to “+a” (red). This means that as values become less negative, a greater glow or tan component arises. For the b* scale, the “−b” (blue) to “+b” (yellow) indicates shift to the yellow component of glow or tan. Accordingly, the pre-heated sample (45° C.) more rapidly generates the equilibrium coloration (glow or tan) than that of the cold lotion sample. More particularly, most of the coloration with the pre-heated sample has occurred at 5 minutes. This same level of coloration requires at least 10 minutes from the cold lotion sample. Equilibrium for both is reached at 20 minutes.

What is claimed is:

1. A skin tanning product dispensed from a heating device comprising:
(i) a cosmetic composition comprising from about 0.1 to about 20% by weight of a tanning agent in a cosmetically acceptable carrier;
(ii) a package containing the cosmetic composition; and
(iii) instructions associated with the package describing use of the cosmetic composition which comprises charging the cosmetic composition into the heating device, applying heat to the composition, thereafter activating a dispensing mechanism associated with the device and transferring dispensed heated composition to a human body.

2. The product according to claim 1 wherein the tanning agent is dihydroxyacetone.

3. The product according to claim 1 wherein the tanning agent is a combination of dihydroxyacetone and erythrulose in a relative weight ratio of 6:1 to 1:2.

4. The product according to claim 1 wherein the heated composition is at a temperature from 38° to 54° C.

5. A method for tanning or imparting glow to skin comprising:
(i) providing a cosmetic composition comprising from about 0.1 to about 20% by weight of a tanning agent in a cosmetically acceptable carrier;
(ii) providing a heating device comprising a chamber for receiving the cosmetic composition, a heating element for imparting heat to the cosmetic composition, and an outlet for dispensing heated cosmetic composition; and
(iii) dispensing from the heating device and applying the heated cosmetic composition onto the skin.

6. The method according to claim 5 wherein the tanning agent is dihydroxyacetone.

7. The method according to claim 5 wherein the tanning agent is a combination of dihydroxyacetone and erythrulose in a relative weight ratio of 6:1 to 1:2.

8. The method according to claim 5 wherein the ratio is 4:1 to 2:1.

9. The method according to claim 5 wherein the cosmetic composition is contained in a package, and the method further comprises associating instructions with the package describing use of the cosmetic composition which comprises charging the cosmetic composition into the heating device, applying heat to the composition, activating a dispensing mechanism associated with the device and transferring dispensed heated composition to a human body.

10. The method according to claim 5 wherein the heated cosmetic composition is at a temperature from 38° to 54° C.
